(19) United States
(12) Patent Application Publication
(10) Pub. No.: US 2014/0090652 A1
(43) Pub. Date: Apr. 3, 2014

(54) INTRAORAL DEVICE FOR TREATMENT OF SNORING, SLEEP APNEA AND TMD

(71) Applicant: FARHAD HAKIMI, NEW YORK, NY (US)

(72) Inventor: FARHAD HAKIMI, NEW YORK, NY (US)

(21) Appl. No.: 13/633,171

(22) Filed: Oct. 2, 2012

(51) Int. Cl. A61F 5/56

(52) U.S. Cl. 128/848

(57) ABSTRACT

The invention is a device for removable placement in the mouth, which advances the lower jaw and draws the tongue forward and upward, thereby providing relief for snoring and obstructive sleep apnea. The device includes an upper portion, the upper surface of which is adapted for engagement with the user's upper dentition. A downward extension, suspended from the anterior part of the upper portion, features an anterior surface that engages the back of the lower front teeth, so as to advance the lower jaw forward with respect to the upper jaw. The posterior surface of the downward extension features a rearward extension, which is curved upward at its lateral margins to meet the upper portion, so as to define, with the hard palate of the user, a cavity that is sized and shaped to snugly engage the user's tongue. Within the defined cavity, the device preferably features a soft elastomeric surface.

### FIELD OF THE INVENTION

This invention relates to intraoral devices designed to position the tongue and jaw so as to prevent and treat snoring and sleep apnea.

St. Matthew's Church Background

Obstructive Sleep Apnea (OSA) is intermittent airflow blockage, which occurs when the tongue and other soft tissue move backwards and block the airway during sleep. Numerous such blockages during the night produce intermittent oxygen deprivation, leading to waking and poor quality sleep. An estimated 20 million Americans experience some degree of obstructive sleep apnea. Snoring is a related and much more common problem, caused by narrowing of the airway to the point that the speed of air across the soft palate causes aeroelastic fluttering, a vibration of the soft tissue that can be loud enough to disturb the sleep of the patient and his or her sleeping partner.

The economic and social costs of OSA and chronic snoring are significant. Potential harmful effects, beyond disrupted sleep and chronic fatigue, include morning headaches, irritability, brain damage, cognitive dysfunction, impotency, high blood pressure, diabetes, heart attacks, and congestive heart failure. Indirect effects of chronic fatigue include inefficient or poor work, motor vehicle crashes and workplace accidents, which in turn can cause serious injury and death.

A variety of techniques for treating obstructive sleep apnea and snoring have been developed. Among the more common are Continuous Positive Airway Pressure (CPAP); surgical removal of soft tissue in the throat; surgical advancement of the jaw or tongue; pharmaceuticals; and jaw- and tongue-positioning orthotic devices. Positioning orthotic devices have used suction, friction, magnets, tongue piercing, and a wide variety of external body positioning structures to hold the jaw or tongue in place and/or urge it away from the airway.

Continuous Positive Airway Pressure (CPAP) is a means of keeping the airway open by constant positive air pressure, which is provided by a machine which continuously pumps air into a mask that the sleeper wears on his or her face. The positive air pressure keeps the airway open. CPAP is first-line therapy for a large percentage of people who are diagnosed with obstructive sleep apnea.

CPAP has several disadvantages. The mask can be uncomfortable, it is difficult to maintain a good seal between the mask and the face, and it limits freedom of movement during sleep. The positive airway pressure can have a negative effect on spontaneous respiration, dry out nasal passages, and cause congestion, headaches, facial distortion, abdominal bleeding, nosebleeds, and chest discomfort. The noise of the machine can disturb the patient or others in the same room, and the machine itself is expensive and inconvenient to travel with. For these and other reasons, only a fraction of the people who are prescribed CPAP as therapy consistently use it.

One invasive approach to treating obstructive sleep apnea is opening of the airway by surgical removal of some of the throat tissues involved in airway narrowing and vibration, for example by removal of a portion of the soft palate, uvula, or tongue. Another invasive approach involves surgically moving the interfering structures and tissues, typically the jaw and tongue, forward and away from the airway. In addition to being highly invasive, irreversible, and costly, surgery has a relatively high failure rate. There is considerable pain and discomfort for the patient, and a long recuperative period. Undesirable side effects may include speech impairment and difficulties in swallowing.

External devices intended to position the body and/or jaws, such as chin straps, head straps, neck braces, and various pillows have been designed and marketed, with little success. Pharmaceutical treatments intended to affect the respiratory system, or the relevant musculature, have likewise met with limited success.

Jaw-positioning orthotics generally attach to the upper and lower teeth, and hold the lower jaw forward, to encourage forward movement of the tongue relative to the soft palate. Jaw-positioning orthotics can cause temporomandibular joint (TMJ) pain (a.k.a. TMJ disorder, TMD), and can be uncomfortable to wear, which limits patient compliance. Long term use can alter the normal bite relationship of the dental arches.

However, due to their relatively low cost, ease of use, and ready reversibility, jaw-positioning orthotics remain a popular option, and this particular art has been the subject to a great deal of patent activity. Recent examples include U.S. Pat. Nos. 7,451,767, 7,730,890, 7,730,891, 7,861,724, 7,954,494, and 8,132,567, and U.S. Patent Application Nos. 2005/0150504, 2007/0028926, 2008/0223367, 2009/0178684, 2010/0065067, 2010/0083971, 2010/0242970, and 2012/0060848. The majority of the prior art orthotics employ a two-piece design, with separately formed upper and lower elements for engaging the upper and lower dentition, connected by a wide variety of hinges and adjustable linkages.

There are also oral orthotics designed to position or move the tongue up, down, or forward, most often using suction or friction to hold the tongue, or in some cases tongue-piercing studs held by mechanical or magnetic means, and there are orthotics which seek to protect the airway from incursion by the tongue. Recent examples include U.S. Pat. Nos. 6,055,986 6,244,865, 6,295,988, 6,408,852, 6,467,484, 6,494,209, 6,766,802, 6,976,491, 7,073,506, 7,137,393, 7,328,698, 7,607,439, 7,770,582, 8,091,554, and 8,122,889, and U.S. Patent Application Nos. 2005/0166929, 2009/0126742, 2011/0048431, 2011/0120476, 2011/0155143, 2011/0226264, 2011/0259346, 2011/0297162, 2011/0315149, and 2012/0073581.

Oral orthotics intended to position both the lower jaw and the tongue have less often been the object of inventors; recent examples are U.S. Patent Application Nos. 2011/0232651 and 2012/0103345.

All of the prior art approaches to treating obstructive sleep apnea are of limited effectiveness and/or have distinct disadvantages; this is reflected by the approximately 1,500 U.S. patents and applications filed for methods and devices directed to the relief of OSA and snoring. Obstructive sleep apnea remains a widespread condition, with serious health consequences and inadequate treatment options. There remains a need for non-invasive and more effective treatments for obstructive sleep apnea which overcome at least some of the disadvantages of the prior art solutions.

### SUMMARY OF THE INVENTION

This invention is a removable intra-oral orthotic device, which both advances the lower jaw and draws the tongue forward and upward, thereby providing relief for snoring and obstructive sleep apnea. The device includes an upper portion, the upper surface of which is adapted for engagement with the user's upper dentition and, optionally, the user's hard palate. A downward extension, suspended from the anterior portion of the device, features an anterior surface that engages the back of the lower front teeth. Engagement of this exterior anterior surface with the user's lower front teeth advances the user's jaw forward with respect to the user's upper jaw. From this point, the downward extension curves back to form a rearward extension, which is curved upward at its lateral margins so as to form, with the inner surfaces of the upper portion, a cavity that is sized and shaped to snugly engage the user's tongue. The concave surface of the rearward extension is preferably provided with a layer of soft elastomer, and optionally presents a roughened texture for attracting and positioning the tongue in a forward position.

The device of the invention does not restrict sleeper movement by connection with an external hose and machine, does not generate noise, and is easily portable. Unlike surgical procedures, the device of the invention is non-invasive and readily reversible, with essentially no risk of pain, speech impairment, or swallowing problems. This device controls the position of the tongue directly, via a passive vacuum, rather than through a large movement of the jaw, and it does not lock the jaws together in a fixed relationship, thereby minimizing the likelihood of TMJ/TMD. The tongue is drawn forward and upward, to a natural position behind the front incisors, which permits the user to swallow without disengaging the tongue from the appliance. Over time, with continued use of the device, the tongue and associated musculature are stimulated and conditioned to assume a more desirable forward and upward position within the mouth.

Unlike devices that use active mechanical suction to hold the tongue in place, the invention does not create a risk of edema or tissue damage, and it allows sufficient tongue movement to provide a degree of comfort to the user. The user breathes normally through the nose when the device is in place, rather than through the mouth, which lessens the likelihood of the tongue falling back into the throat. Finally, the device of the invention helps to open all three sections—upper, middle, and lower—of the airway.

The orthotic device of the invention is a simple one-piece design, with no moving parts and nothing to adjust. In preferred embodiments, it employs a combination of hard materials, for structural integrity, and soft materials for comfortable and non-injurious contact with the tongue and other soft tissues of the mouth.

The device of the invention is suitable for the treatment and/or relief of sleep apnea, snoring, upper airway resistance syndrome, certain forms of TMJ/TMD, and any other conditions that benefit from advancement of the lower jaw and/or tongue.

### BRIEF DESCRIPTION OF THE FIGURES

FIG. 1 is a drawing of the human airways in the mid-saggital plane.

FIG. 2 is a drawing of the human airways, with a device of the invention in place.

FIGS. 3A-3C are perspective drawings of a device of the invention.

FIG. 4 is a cross-section of a device of the invention.

FIG. 5 is a cross-section of a device of the invention having an elastomer layer.

### DETAILED DESCRIPTION OF THE INVENTION

The present invention is an improvement on the dental prosthetic devices disclosed in U.S. Pat. No. 5,915,385, the entire contents of which are incorporated herein by reference. The invention provides a device for removable placement in the mouth of a user for the relief of sleep-related breathing disorders, which comprises (a) an upper portion adapted to be engaged with the upper dentition of a user; (b) a downward extension suspended from the anterior end of the upper portion, the anterior surface of said extension being configured to be engaged against at least some of the lower front teeth in the user's mouth, causing the jaw of the user to be urged forward; which curves rearward to form (c) a rearward extension, having an upper surface which is curved upward at its lateral margins so as to define, with the hard palate of the user, a tongue-receiving cavity that is sized and shaped to engage the tip and a small portion of the blade of the user's tongue.

Preferably, the cavity is sized and shaped to engage at least the first anterior centimeter of the user's tongue, and more preferably at least the first two centimeters. In preferred embodiments, the upper surface of the upper portion is provided with inner and outer upstanding wall portions extending upwardly from the upper surface, so as to define a channel for receiving at least a portion of at least some teeth of the upper dentition in the user's mouth.

Preferably, the upper surface of the rearward extension is covered with a soft, dimensionally stable elastomer, such as for example a crosslinked organopolysiloxane. The upper surface may optionally be provided with a bioadhesive material to help retain the tongue in the desired position.

In preferred embodiments, the portion of the device that contacts the tip of the user's tongue is provided with a textured surface, having sufficient roughness to attract the tongue.

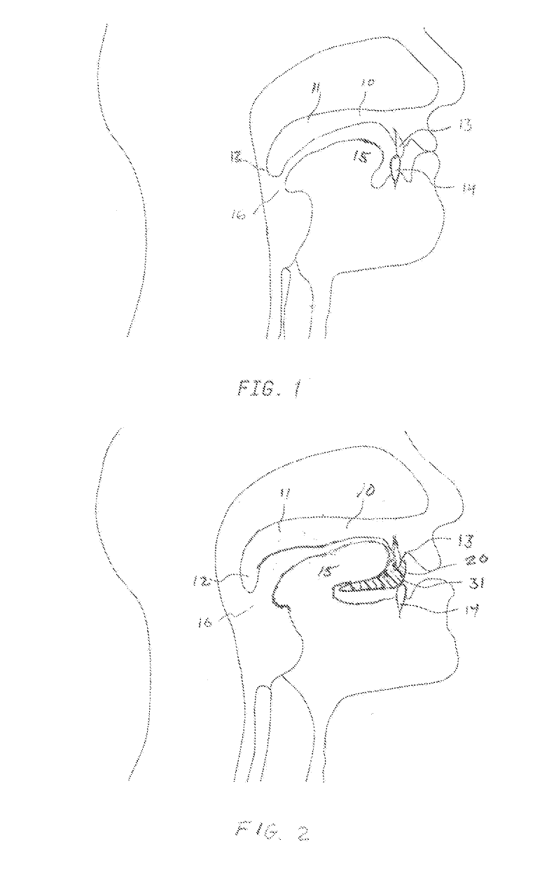

FIG. 1 shows the human airway in cross-section, with the mouth closed and the upper and lower dentition in their normal relative positions. Relevant features are the hard palate 10, soft palate 11, uvula 12, upper incisor 13, lower incisor 14, and tongue 15. Behind the tongue is the upper airway 16.

FIG. 2 also shows the human airway in cross-section, but with a device 20 according to the present invention in place. The device fits snugly over the upper dentition, which includes upper incisor 13, thereby fixing the position of the device with respect to the hard palate and maxillary arch. The lower incisor 14 is displaced in a forward direction by the anterior surface 31 of the device, thereby advancing the mandible and the tongue and opening the upper airway 16. The tongue fits closely in the cavity defined by the hard palate and the rearward extension 42 of the device.

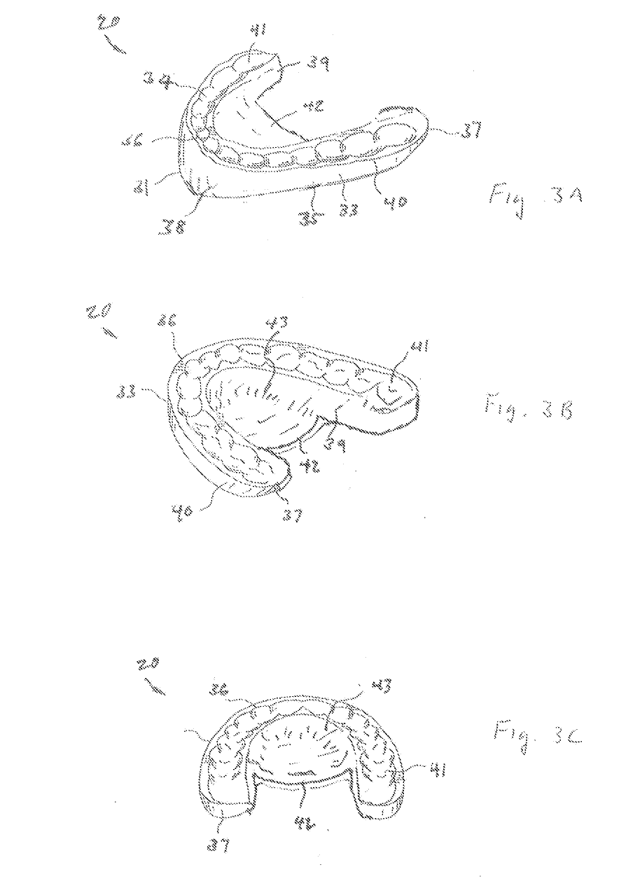

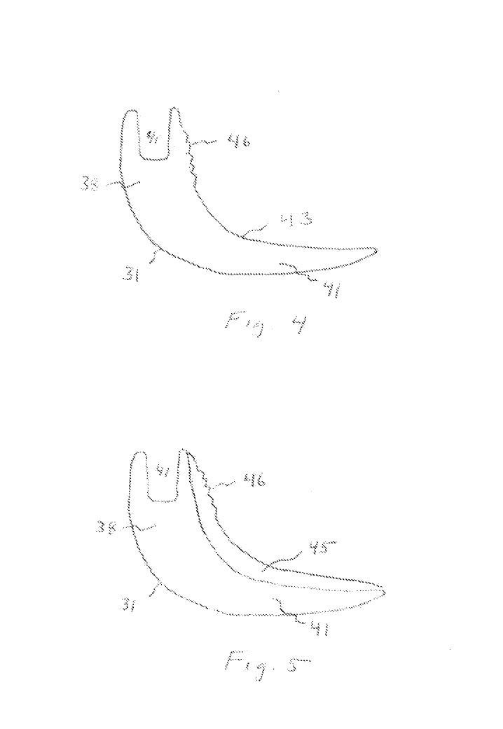

As shown in FIGS. 3A-3C, the device 20 comprises an upper portion 33 advantageously configured as a u-shaped mouthpiece for engaging at least some of the teeth of the upper dentition of a patient or user (not shown). In a preferred embodiment, the entire device 20 is formed from a resilient polymeric material. Suitable materials include, but are not limited to, thermoplastic transparent heat-curing acrylic resins such as a polymethylmethacrylate (PMMA), rubber modified acrylics, vinyl acrylic copolymers, hydrophilic acrylics, a glycol-modified poly(ethylene terephthalate) such as that available under the trademark KODAR™ PETG copolyester 6763 (Eastman Chemical Products, Inc., Kingsport, Tenn.), and urethane polymers, such as the visible-light-curable urethane dimethacrylate polymer available under the trademark TRIAD™ (Dentsply International Inc., York, Pa.).

Upper portion 33 is provided with upper and lower surfaces 34 and 35 respectively. Upper portion 33 also includes front and rear ends 36 and 37, respectively, and a downward extension 38 attached to upper portion 33 at front end 36.

Inner and outer wall portions, 39 and 40 respectively, extend upwardly from inner and outer edges of upper surface 34. Inner and outer wall portions 39 and 40 extend forward from both the left and right rear ends 37 towards the front end 36, where the left and right sections join, to form a U-shaped trough 41. The trough is designed to fit the arch of the maxilla. Trough 41 is defined between inner and outer wall portions 39 and 40, and in one embodiment of the invention, may be custom-formed to fit closely upon at least some of the maxillary (upper dentition) teeth. In another embodiment, the walls of the trough may be sufficiently spaced apart to receive an uncured resin as well as the maxillary teeth. By partially filling trough 41 with an uncured resin, such as a fast-setting acrylic resin, and impressing the maxillary teeth into the resin, upper portion 33 may be custom-fitted to the user's upper dentition. Once the resin is cured, the impression is fixed, and the device 20 is then capable of being releasably secured to the user's upper dentition when properly aligned and pressed into position thereon.

As further illustrated in FIG. 3A, upper portion 33 includes an anterior downward extension 38 attached at the front end 36. Anterior extension 38 depends downward from front end 36 of upper portion 33 and varies in size and thickness according the user to which device 20 is fitted. Downward extension 38 features an anterior surface 31, for engagement with the back and/or upper edge of the user's lower incisors, and then curves back to become a rearward extension 42 which forms a concave platform on which the user's tongue can rest.

During the fitting of device 20, discussed in detail below, anterior surface 31 of the downward extension 38 is configured so as to conform to the interior (lingual surface) of the front teeth of the lower dentition. Based on an examination of the user's jaw by a dental professional, the downward extension 38 is custom configured in both orientation and thickness, such that when device 20 is properly inserted and fitted within the mouth, downward extension 38 engages the user's lower dentition so as to maintain a forward posture of the user's lower jaw relative to the upper jaw, as shown in FIG. 2, such that the volume of the user's upper airway is increased so as to alleviate snoring and apnea. For maximum comfort, exterior surface 31 of the downward extension 38 is preferably formed with a smooth surface, to prevent abrasion of the user's teeth and tissues when device 20 is properly positioned in the user's mouth.

The upper surface 43 of rearward extension 42 curves upward at its front and lateral edges to meet and smoothly blend into the inner wall 39. Extension 42 accordingly features an upper, concave surface 43. As shown in FIG. 4, the combination of upper surface 43, inner wall 39, and the user's hard palate 10, together define a tongue receiving cavity 44 shaped to fit the blade of the user's tongue. When device 20 is properly positioned upon a user's upper dentition, the concave surface 43 attracts the user's tongue and maintains the tongue in a raised and forward position, by a combination of frictional attraction between the tongue and concave surface 43, and, in preferred embodiments, a vacuum formed by the expulsion of air from receiving space 44 when the tongue is inserted. The positioning of the tongue in a forward position increases the distance between the tongue and the uvula and soft palate; this forward positioning of the tongue complements the shifting of the lower jaw forward relative to the upper jaw, and assists in increasing the volumetric capacity of the user's upper airway. This prevents apnea and also reduces turbulent airflow, lowering the possibility of vibration of the uvula and soft palate.

In one embodiment of the invention, the portion of concave surface 43 and/or inner wall 39 which will be in contact with the tip of the tongue is provided with a textured surface 46, having sufficient roughness to be noticeable to the user. Humans have a natural tendency to reflexively probe rough surfaces within the mouth with the tongue, and the presence of the textured surface therefore serves to attract the tongue into the desired position. By reducing the need for the user to consciously attend to the tongue's position, this feature makes it easier for the user to drift off to sleep.

As shown in FIG. 5, concave surface 43 is preferably coated with a layer 45 of a soft elastomer. As used herein, “soft” indicates a Shore A Durometer value of about 40 or less. Organopolysiloxane elastomer coatings are preferred, as they are characterized by high elongation and softness, but still exhibit dimensional stability and low water absorption. The use of such elastomers in the making of dental orthotics is disclosed in U.S. Pat. No. 5,224,958, which is incorporated by reference herein in its entirety. Suitable organopolysiloxanes include, but are not limited to, copolymers of dimethyl and methylvinyl siloxanes, preferably those having from about 99.90 to about 99.99 mole percent dimethyl siloxane units and from about 0.01 to about 0.10 mole percent methylvinyl siloxane units. Suitable examples of such materials are commercially available under the trademark SILASTIC™ (Dow Corning, Midland, Mich.), and under the “Realistic” brand (Factor II Inc., Lakeside Ariz.) These materials are supplied by the manufacturers as two-component kits, having separate silicone and platinum-based catalyst components which the practitioner can blend together manually, shape or mold to the desired form, and then thermally cure to form the desired elastomer.

The preferred SILASTIC™ silicone is that designated Q7-4720, which is a copolymer comprising approximately 99.95 to about 99.97 mole percent dimethyl siloxane and approximately 0.03 to about 0.05 mole percent methylvinyl siloxane. When prepared and cured according to directions, the resulting elastomer exhibits a Shore A Durometer of about 19 to about 27 as determined by ASTM D 2240. The preferred “Realistic” silicone is that designated A-588-2, which yields an elastomer having Shore A Durometer of about 20.

In a preferred embodiment, a reactive primer is painted onto the concave surface 43 of the device, and onto the inner surface of inner wall 39, either neat or as a solution in a suitable solvent such as naphtha, toluene, or MEK, prior to applying the organopolysiloxane layer. For bonds of optimum strength, more than a single coat may be applied, with the coating being allowed to dry between applications. Most preferably, the primer-painted device is oven-dried above ambient temperature after the final coating of coupling agent has been applied, so as to drive off residual solvent.

Suitable primers include, but are not limited to, such commercially available silanes as methyltriacetoxysilane, vinyltriacetoxysilane, methyltris-(methylethylketoximine)silane, vinyltris(methylethylketoximine)silane tetra(n-propoxy)silane, and tetra(2-methoxyethoxy)silane, with additives such as methyl methacrylate and tetrabutyl titanate. Preferred primers are those sold commercially for the purpose, such as the A-304, A-306, and A-330-G “platinum primers” (Factor II Inc., Lakeside Ariz.)

Additionally, or alternatively, concave surface 43 may be provided with a layer of bioadhesive material to assist in retaining the tongue in the forward position while the user is asleep. Suitable bioadhesive materials include, but are not limited to, those comprising natural polymers, semisynthetic polyhydric polymers, synthetic polyhydric polymers or polycarboxylic acid polymers, hydrocolloids, or combinations thereof. Examples of polyhydric polymers which can be used include hydroxypropyl cellulose, hydroxypropyl methylcellulose, hydroxyethylcellulose, carboxymethyl cellulose, dextran, pectin, alginate, arabinogalactan, pullulan, gaur-gum, hyaluronic acid, pectins, derivatized starches, acrylic acid polymers, polymers of acrylic acid esters, acrylic acid-ester copolymers, polymers of vinyl alcohols, alkoxyvinyl polymers, chitosan, polyethylene oxide polymers, and polyethers, as well as a variety of combinations thereof. The bioadhesive may further comprise natural gums and gels, such as karaya gum, ghatti gum, tragacanth gum, xanthan gum, jaraya gum and the like, as well as seed gums such as guar gum, locust bean gum, psillium seed gum and the like, and hydrocolloid gels prepared from polysaccharides extracted from Fronia elephantum, Sapindus trifoliatus, Kunjac, and cashew.

Preferred bioadhesives are the crosslinked polyacrylic acids sold under the trade names CARBOPOL™and NOVEON™, such as CARBOPOL™ 940, 934, 934P, 941, 970, and 974, and NOVEON™ AA-1 (Lubrizol Corp., Wickliffe, Ohio) The bioadhesive material may optionally be provided with sweeteners and/or flavors, to provide a pleasant and/or refreshing taste in the use's mouth.

For optimal fitting and proper placement within a user's mouth, fabrication of device 20 is advantageously performed in dental laboratories by skilled technicians, who operate from an impression made by the clinician who is treating the user. Device 20 may alternatively be fabricated and fitted by a dental professional within a dentist's office, without the need for sending the mold or cast to a dental laboratory.

In one embodiment of the invention, the device 20 is initially obtained by modification of a one-size dental orthosis. Although mouth and dentition shapes and sizes vary considerably, a one-size dental orthosis is initially formed to be of such size and shape as to be capable of deformation to conform with most adult mouths, with minimal further fitting. The one-size dental orthosis may easily be trimmed with scissors or other cutting implements to provide for an optimum fit. Typically, the material of which device 20 is formed, in accordance with this embodiment, enters a soft moldable state when heated in boiling water, but reverts to a pliant but resilient state upon cooling. Thus, device 20 can be molded in situ to conform to the user's upper dentition and lower jaw configuration.

Upon positioning the user's upper teeth within trough 41, anterior portion 38 may be configured with the proper thickness and inclination to provide the desired amount of forward shift of the lower jaw. After forming and fitting, device 20 is removed from the user's mouth to permit drying and hardening. Minor adjustments to the shape and form of the device may then be made by cutting and trimming in the usual manner.

Dental polymers like PMMA may contain polymerization initiators, such as ABIN or benzoyl peroxide. The presence of such initiators in the polymer may interfere with the catalyst system of the silicone elastomer, in which case the elastomer may not fully cure. For this reason it is preferred that the hard polymeric substrates of the composite structure be substantially free of residual initiators. This may usually be accomplished by heating the device for at least ten minutes in boiling water or in a microwave oven. The device should be subjected to final shaping and trimming prior to silicone bonding.

The device is held, preferably with forceps or cotton gloves, while the surfaces to be bonded are cleaned with a gauze moistened with acetone. At least one coat of primer is applied according to the manufacturer's instructions, and allowed to dry. The silicone precursors are mixed according to the manufacturer's instructions, and the mixture is applied in a thick layer to the primed surfaces of the device. A tongue-shaped form, or preferably a model of the actual patient's tongue, is seated in the silicone, and the arrangement is clamped and slowly heated to the curing temperature in a dry oven. The silicone is then thermally cured according to the manufacturer's instructions. In the case of the “Realistic” A-588-2 silicone, for example, curing may be done for one hour at 210 ° F., or for three hours at 180 ° F.

Device 20 may be marketed to dental professionals in a the form of a kit: a package which contains a variety of pre-formed devices which may then be fitted to the user's upper dentition and custom sized for optimum fit. To provide for the varying mouth shapes and sizes, dental professionals may purchase packages provided with two or three devices of different sizes. The kit provides a deformable resin, such as a fast-setting acrylic resin, which is filled within trough 41. Engagement of the user's upper dentition within the resin then forms an impression of the user's upper dentition, to provide for a custom fit. After the resin hardens, the dental professional proceeds to form device 20 to further conform to the user's mouth by cutting and trimming any necessary portions. In order to properly fit device 20 to a child, for example, posterior end 37 may need to be trimmed or cut away to provide the proper size for engaging the teeth of the child's upper dentition.

FIGS. 1 and 2 illustrate the upper and lower dentition, jaw position and soft tissues of the mouth. FIG. 1 shows the normal dentition and jaw positioning of an average person. It can be seen that in this normal positioning, the teeth of the upper and lower dentition form an “overbite” arrangement. FIG. 1 also illustrates the natural positioning of the tongue 15, uvula 12, hard palate 10 and soft palate 11. As shown, soft palate 11 extends rearwardly and terminates in the uvula 12. The dimensions of the upper airway opening 16 are normally defined by the relative positions of the soft palate 11, uvula 12 and tongue 15.

FIG. 2 illustrates an embodiment of device 20 in situ. The teeth of the upper dentition, represented by incisor 13, are positioned within a channel on device 20. When the user's mouth closes naturally, anterior downward extension 38 engages the rear and/or upper edge of incisor 14. As a result of the inclination of extension 38, the engagement of the anterior surface against the lower dentition urges the user's jaw forward relative to the upper jaw. In preferred embodiments, the spacing between the upper dentition and the lower dentition created by device 20 is such that the lips remain closed, so as to discourage inhalation and exhalation though the mouth. Should breathing through the mouth be desirable for any reason, however, the spacing may be increased appropriately.

The result of the combined engagements of anterior extension 38 against the teeth of the user's lower dentition, and the upward and forward positioning of the user's tongue 15 above device 20, is an increase in the dimensions of the of the upper airway opening 16. The resulting increase in volume reduces air velocity, which in turn reduces the associated vibration or fluttering of the soft tissues. As a result of the increased spacing, and the reduction in soft tissue vibration, apnea and snoring may be substantially reduced or eliminated.

When device 20 has been properly fitted, it securely adheres to the user's upper dentition when properly inserted and positioned. When the user lies down in bed in a supine position the lower jaw exists in an unstressed position, such that the anterior portion 38 naturally engages the interior surface of the user's lower dentition. The engagement of the user's lower teeth with the sloped extension 38 induces the lower jaw to extend forward relative to the upper jaw. At the same time, the user's tongue is attracted to the concave surface of the device, which as discussed previously may be provided with a bioadhesive material to assist in retaining the tongue in a forward position, so that the distance between the tongue and the uvula and soft palate is increases. This maintenance of the user's tongue in a forward and upward position further increases the opening of the upper airway to facilitate breathing and reduce vibration of the uvula and soft palate.

Although the above-described device is adapted for and directed to the reduction and elimination of snoring and obstructive sleep apnea, device 20 may also function as a stress-reducing device, by relieving pressure on the temporomandibular joint. For relieving stress, the primary objective of device 20 is to separate the upper and lower dentition and shift the lower jaw slightly forward, and less attention needs to be given to the bioadhesive, silicone layer, textured surfaces, and other tongue-positioning aspects of the device, any or all of which may be omitted.

The examples provided above are intended to serve only as illustrations of specific embodiments of the invention, and accordingly the invention is not limited to the examples and drawings provided. It will be understood that various omissions, substitutions and changes in the form and details of the disclosed examples of the invention may be made by those skilled in the art, without departing from the spirit of the invention. Applicant's exclusive rights in the invention are limited only by the scope of the claims appended hereto, and the equivalents thereof

I claim:

1. A device for removable placement in the mouth of a user for the relief of sleep-related breathing disorders, comprising:
(a) an upper portion adapted to be engaged with the upper dentition of a user;
(b) a downward extension suspended from the anterior end of the upper portion, the anterior surface of said extension being configured to be engaged against at least some of the lower front teeth in the user's mouth, wherein engagement of the anterior surface with the lower front teeth causes the jaw of the user to be urged forward; and
(c) a rearward extension, extending from the downward extension, having an upper surface which is curved upward at its lateral margins so as to define, together with the hard palate of the user, a tongue-receiving cavity that is sized and shaped to engage the tip and at least the first anterior centimeter of the user's tongue.

2. The device according to claim 1, wherein the cavity is sized and shaped to engage at least the first two anterior centimeters of the user's tongue.

3. The device according to claim 1, wherein the upper surface of the upper portion is provided with inner and outer upstanding wall portions extending upwardly from said upper surface, so as to define a channel for receiving at least a portion of at least some teeth of the upper dentition in the user's mouth.

4. The device according to claim 2, wherein the upper portion is provided with inner and outer wall portions extending upwardly from the upper portion, so as to define a channel for receiving at least a portion of at least some teeth of the upper dentition in the user's mouth.

5. The device according to any one of claims 1-4, wherein the upper surface of the rearward extension is covered with a soft, dimensionally stable elastomer.

6. The device according to claim 5, wherein the soft elastomer is a crosslinked organopolysiloxane.

7. The device according to any one of claims 1-4, wherein the upper surface of the rearward extension is provided with a bioadhesive material to retain the tongue therein.

8. The device according to claim 5, wherein the upper surface of the rearward extension is provided with a bioadhesive material to retain the tongue therein.

9. The device according to claim 6, wherein the upper surface of the rearward extension is provided with a bioadhesive material to retain the tongue therein.

10. A device for removable placement in the mouth of a user for the relief of sleep-related breathing disorders, comprising:
(a) an upper portion adapted to be engaged with the upper dentition of a user;
(b) a downward extension suspended from the anterior end of the upper portion, the anterior surface of said extension being configured to be engaged against at least some of the lower front teeth in the user's mouth, wherein engagement of the anterior surface with the lower front teeth causes the jaw of the user to be urged forward;
(c) a rearward extension, extending from the downward extension, having an upper surface which is curved upward at its lateral margins so as to define, with the hard palate of the user, a tongue-receiving cavity that is sized and shaped to engage the tip and at least the first anterior centimeter of the user's tongue; and
(d) a textured surface having sufficient roughness to attract the tongue, said textured surface being located at a portion of the device that contacts the tip of the user's tongue.
